(19) United States
(12) Patent Application Publication
(10) Pub. No.: US 2025/0236819 A1
(43) Pub. Date: Jul. 24, 2025

(54) CLEANING COMPOSITION

(71) Applicant: GLAXOSMITHKLINE CONSUMER HEALTHCARE (UK) IP LIMITED, Weybridge, Surrey (GB)

(72) Inventors: Adam Simon HAYWARD, Weybridge, Surrey (GB); Robert LUCAS

(21) Appl. No.: 18/853,889

(22) PCT Filed: Apr. 12, 2023
(86) PCT No.: PCT/EP2023/059571

§ 371 Date: Oct. 3, 2024

(30) Foreign Application Priority Data
Apr. 14, 2022 (EP) 22168580.3
Apr. 14, 2022 (GB) 2205594.1

(51) Int. Cl. C11D 3/39; A01N 25/30; A01N 37/40; A01N 59/00; A01P 3/00; A61L 2/18; A61L 101/02; A61L 101/36; A61L 101/40; A61L 101/44; C11D 1/14; C11D 3/20; C11D 3/48; C11D 17/04

(52) U.S. Cl. CPC C11D 3/3942; A01N 25/30; A01N 37/40; A01N 59/00; A01P 3/00; A61L 2/186; C11D 1/146; C11D 3/2079; C11D 3/2086; C11D 3/2096; C11D 3/48; C11D 17/049; A61L 2101/02; A61L 2101/36; A61L 2101/40; A61L 2101/44

(57) ABSTRACT

The present invention relates to cleaning compositions comprising low levels of hydrogen peroxide and at least one acid selected from the group consisting of salicylic acid, furoic acid and formic acid. The compositions and methods of the invention are particularly suitable for use in household cleaning, for example in hard-surface cleaning or cleaning soft surfaces such as textiles and laundry.

### FIELD OF THE INVENTION

The present invention relates to cleaning compositions comprising low levels of hydrogen peroxide and at least one acid selected from the group consisting of salicylic acid, furoic acid and formic acid. The compositions and methods of the invention are particularly suitable for use in household cleaning, for example in hard-surface cleaning or cleaning soft surfaces such as textiles and laundry.

### BACKGROUND TO THE INVENTION

A large number of compositions for use in cleaning hard and soft surfaces have been described in the art.

Whilst such compositions may provide suitable cleaning performance, there is a need for alternative cleaning compositions for cleaning and/or disinfecting hard surfaces, such as kitchen and bathroom surfaces, and soft surfaces such as textiles, for example laundry.

In particular, it would be beneficial to provide efficient and cost-effective compositions for reducing and/or eliminating microorganisms on soft and hard surfaces that do not rely on harsh chemicals and that may be safer for consumers. There is also a need for compositions that are more environmentally friendly and avoid the need for complex activators such as TAED and the like.

### SUMMARY OF THE INVENTION

The Inventors have developed aqueous cleaning compositions with desirable cleaning properties that may be used for cleaning hard surfaces or soft surfaces.

In a First Aspect, there is provided an aqueous cleaning composition comprising (a) hydrogen peroxide, (b) at least one acid selected from the group consisting of salicylic acid, furoic acid and formic acid, (c) an anionic surfactant and (d) at least 90% by weight of water. The aqueous cleaning composition may be a hard-surface cleanser and/or a soft-surface cleanser. In certain embodiments, the aqueous cleaning composition is an aqueous hard-surface cleanser composition. In certain embodiments, the aqueous cleaning composition is an aqueous soft-surface cleanser composition.

More particularly the aqueous cleaning compositions comprise or consist essentially of: (a) from about 0.05% to about 4% by weight of hydrogen peroxide, (b) from about 0.01% to about 0.25% by weight of at least one acid selected from the group consisting of salicylic acid, fuoric acid and formic acid, (c) from about 0.05% to about 2.00% by weight of an anionic surfactant, and (d) from about 90% by weight of water, wherein the composition has a pH of from 2.8 to 3.5.

Compositions of the Invention are aqueous compositions comprising at least 90% by weight of water. More particularly, the compositions may comprise at least 91%, at least 92%, at least 93%, at least 94%, at least 95%, at least 96%, at least 97%, at least 98% or at least 99% by weight of water.

Exposure of a test suspension of either Candida albicans or Klebsiella pneumoniae (1-5×10⁸ cfu per ml) to a composition of the invention results in at least a log 2.5 reduction in the number of colony forming units within 60 seconds, preferably at least a log 3 reduction, more preferably at least a log 4 reduction, most preferably at least a log 5 reduction within 60 seconds.

The compositions of the invention are storage-stable for a commercially reasonable period of time. More particularly, compositions of the invention are storage-stable for at least 3 months after preparation, particularly for at least 6 months after preparation, more particularly at least 12 months after preparation, yet more particularly at least 24 months after preparation.

Storage stability of compositions of the Invention may be determined using direct measures or indirect measures. Direct measures of storage-stability include measures of physical stability, such as precipitation, and/or chemical stability, such as chemical degradation of one or more of the constituents. Indirect measures of storage-stability include measures of microbial killing, for example determined using a bacterial killing assay, fungal killing assay and viral inactivation or killing assay.

As used herein, the term “storage stable” means that the aqueous mixture components do not precipitate (physically stable) or chemically degrade (chemically stable) over the specified time period.

Compositions of the invention may further comprise (e) a means for adjusting or maintaining the pH of the composition. In certain embodiments the compositions of the invention comprise a means for adjusting or maintaining the pH of the composition within the range of from 2.8 to 3.5.

In some embodiments, compositions of the invention consist essentially of (a) from about 0.05% to about 4% by weight of hydrogen peroxide, (b) from about 0.01% to about 0.25% by weight of at least one acid selected from the group consisting of salicylic acid, furoic acid and formic acid, (c) from about 0.05% to about 2.00% by weight of an anionic surfactant, (d) from about 90% by weight of water, and (e) a means for adjusting or maintaining the pH of the composition, wherein the composition has a pH of from 2.8 to 3.5.

The composition may comprise from about 0.01% to about 0.09% by weight of the means for adjusting or maintaining the pH of the composition. The means for adjusting or maintaining the pH of the composition may be an acid or a buffer. The composition may comprise from about 0.01% to about 0.09% by weight of a buffer. The composition may comprise from about 0.02% to about 0.08% by weight of the means for adjusting or maintaining the pH of the composition. The composition may comprise from about 0.02% to about 0.08% by weight of a buffer. In some embodiments, the means for adjusting or maintaining the pH of the composition is sodium citrate, more particularly, sodium citrate buffer.

Thus, in another embodiment, there is provided an aqueous cleansing composition consisting essentially of: from about 0.05% to about 4% by weight of hydrogen peroxide, from about 0.01% to about 0.25% by weight of an acid selected from the group consisting of salicylic acid, furoic acid and formic acid, from about 0.05% to about 2.00% by weight of an anionic surfactant, from about 90% by weight of water, and from about 0.01% to about 0.08% by weight of a buffer particularly, a sodium citrate buffer, wherein the composition has a pH of from 2.8 to 3.5.

Compositions of the invention may further comprise a sensory component. In certain embodiments, the sensory component is a perfume, a colouring agent and/or a bittering agent.

In further embodiments, there is provided an aqueous storage-stable cleansing composition consisting essentially of: from about 0.05% to about 4% by weight of hydrogen peroxide, from about 0.01% to about 0.25% by weight of an acid selected from the group consisting of salicylic acid, furoic acid and formic acid, from about 0.05% to about 2.00% by weight of an anionic surfactant, from about 90% by weight of water, and from about 0.01% to about 0.08% by weight of a buffer particularly, a sodium citrate buffer, wherein the composition has a pH of from 2.8 to 3.5 and wherein the composition further comprises from about 0.01% to about 0.5% by weight of a sensory component.

In some embodiments the anionic surfactant is a sodium alkyl sulfate. In certain embodiments, the anionic surfactant is a sodium alkyl sulfate selected from the group consisting of sodium lauryl sulfate, sodium lauryl sulfonate and sodium laureth sulfate.

Thus, there is also provided an aqueous storage-stable cleansing composition consisting essentially of: from about 0.05% to about 4% by weight of hydrogen peroxide, from about 0.01% to about 0.25% by weight of at least one acid selected from the group consisting of salicylic acid, furoic acid and formic acid, from about 0.05% to about 2.00% by weight of an anionic surfactant selected from the group consisting of sodium lauryl sulfate, sodium lauryl sulfonate and sodium laureth sulfate, from about 90% by weight of water and from about 0.01% to about 0.08% by weight of a buffer particularly, a sodium citrate buffer, wherein the composition has a pH of from 2.8 to 3.5 and wherein the composition further comprises from about 0.01% to about 0.5% by weight of a sensory component.

In some embodiments, compositions of the invention comprise from about 0.1% to about 2% by weight of hydrogen peroxide, particularly from about 0.25% to 1% by weight of hydrogen peroxide.

Preferably, the acid is salicylic acid. In some embodiments, compositions of the invention comprise from about 0.01% to 0.25% by weight of salicylic acid, particularly from about 0.1% to 0.2% by weight of salicylic acid.

In some embodiments, compositions of the invention comprise from about 0.25% to about 1% by weight of the anionic surfactant. In some embodiments, compositions of the invention comprise from about 0.25% to about 1.00% by weight of an anionic surfactant selected from the group consisting of sodium lauryl sulfate, sodium lauryl sulfonate and sodium laureth sulfate

Compositions of the invention are ready to use and do not require dilution or reconstitution. The compositions may be applied to a surface by pouring, spraying and/or wiping. The compositions are foamable and may be applied to a surface in the form of an aerated foam. Compositions of the invention are substantially free of peracid salts. Compositions of the invention do not comprise TAED.

In some embodiments, the aqueous compositions are hard-surface cleanser compositions. In some embodiments, the aqueous compositions are soft-surface cleanser compositions, for use in cleansing textiles such as laundry. In certain embodiments, the aqueous compositions are laundry detergent booster compositions and/or stain treatment compositions.

In a Second Aspect of the invention, there is provided a container containing the composition of the First Aspect. Particularly, the container is a dispenser. Particularly, the dispenser comprises or consists essentially of, a container defining a reservoir and an applicator head in fluid communication with the reservoir, wherein the reservoir comprises the composition of the Invention and wherein the applicator head is configured to dispense the composition from the reservoir, particularly in the form of a spray and/or foam.

By way of non-limiting example, the container comprises high density polyethylene (HDPE) and/or polyethylene terephthalate (PET). In some embodiments, the container comprises high density polyethylene (HDPE) and polyethylene terephthalate (PET). In some embodiments, the container is a high-density polyethylene (HDPE) container. In some embodiments, the container is a polyethylene terephthalate (PET) container.

In a Third Aspect of the Invention, there is provided a disposable premoistened pad for cleaning hard surfaces comprising: (i) a substrate and (ii) a cleansing composition of the First Aspect, wherein the substrate is impregnated with the composition.

In a Fourth Aspect of the invention, there is provided a hard-surface cleansing composition according to the First Aspect, for use in a method of cleansing a hard-surface.

In some embodiments of the Fourth Aspect, the hard-surface cleansing composition may be provided within a container according to the Second Aspect. In these embodiments, the hard-surface cleansing composition may be applied to the hard-surface by spraying or pouring.

In some embodiments of the Fourth Aspect, the hard-surface cleansing composition may be provided in the form of a substrate impregnated with the cleansing composition according to the Third Aspect. In these embodiments, the hard-surface cleansing composition may be applied to the hard-surface by wiping.

In a Fifth Aspect of the Invention, there is provided a cleansing composition according to the First Aspect, for use in a method of cleansing a soft surface. Thus, the cleansing composition may be a stain treatment composition and/or a laundry detergent booster composition.

In a Sixth Aspect of the Invention, there are also provided methods of cleansing a hard or soft surface comprising maintaining a cleansing composition according to the First Aspect in contact with the hard or soft surface for a period of time.

There is also provided a cleanser composition according to the First Aspect, for use in a method of killing a microorganism, such as Candida albicans, on a hard or soft surface. Particularly, the method comprises, contacting the microorganism on the hard or soft surface with the cleansing composition for a period of time sufficient to kill the microorganism.

Particularly, the microorganism is a bacteria, fungi or virus. In some embodiments, the microorganism is a bacterium, particularly a gram-negative bacterium, such as Klebsiella pneumoniae. In some embodiments, the microorganism is a fungus, particularly a yeast, such as Candida albicans. In some embodiments, microorganism is a virus such as an enveloped or non-enveloped virus, for example an influenza virus or a coronavirus. In certain embodiments the virus is influenza virus H1N1. In certain embodiments the virus is a coronavirus such as SARS-CoV, SARS-CoV-1 or SARS-CoV-2.

The compositions and methods described herein are particularly useful for treating hard surfaces, such as floors, kitchen surfaces, bathroom surfaces and walls.

The compositions and methods described herein are particularly useful for treating soft surfaces such as textiles and laundry, for example as an additive to laundry wash liquor.

### DESCRIPTION OF FIGURES

FIG. 1: Shows a ceramic tile stained with tea comparing an untreated section (UT) with a section treated with a composition of the invention (T).

### DETAILED DESCRIPTION OF THE INVENTION

The present invention is directed towards aqueous cleansing compositions comprising either salicylic acid, furoic acid or formic acid in combination with hydrogen peroxide and an anionic surfactant.

Surprisingly, the Inventors have discovered that such compositions exhibit superior anti-microbial efficacy. Without wishing to be bound by theory, there appears to be a synergistic anti-microbial effect when micro-organisms are exposed to a combination of hydrogen peroxide and the acid component (salicylic, formic or furoic acid) achieving (in a killing assay) log 2.5 or even greater reductions in the number of colony forming units within 60 seconds, even at low levels of hydrogen peroxide. It should be noted that other acids, such as phosphoric or citric acid, do not exhibit such synergistic anti-microbial efficacy and the effect is not linked to pKa, for example.

Advantageously, such compositions have excellent dermal tolerance making them particularly suitable for use in cleansing surfaces where product contact with the skin of the hands, fingers or feet is likely.

Reference to “cleaning” and “cleansing” refers generally to the treatment of a surface to reduce or remove unwanted material such as dirt, oil, stains or microorganisms. The term “hard surface” refers to solid, substantially non-flexible surfaces such as counter tops, tiles, floors, walls, plumbing fixtures, kitchen and bathroom furniture and the like. The term “soft surface” refers to softer, highly flexible materials such as woven and unwoven textiles, for example clothes, fabrics and carpet.

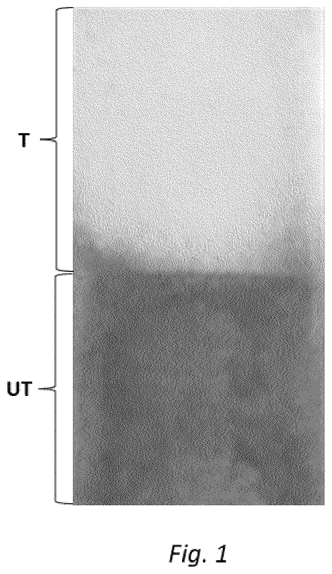

The compositions are effective at cleaning hard surfaces and removing stains (FIG. 1). They are highly surface compatible such that they do not corrode or tarnish plastic, such as acrylic resin, nylon, porcelain, resin, metal or other materials often found in residential homes and commercial environments.

The compositions are also effective at cleaning soft surfaces and removing stains. The compositions may be used for pre-treatment of stains or as laundry additives for use in cleaning textiles as an addition to a laundry detergent in a wash liquor, for example improving (boosting) or adding to the effects of the detergent. When used as a laundry booster, compositions of the invention provide a sanitising or disinfecting effect. These effects may be particularly advantageous when cleaning baby clothes and nappies.

The compositions have a desirable environmental profile breaking down primarily into oxygen and water. More particularly, compositions of the Invention are free of persalts (such as sodium perborate) and commonly used activators (such as TAED, NOBS and LOBS) which may be costly and have environmental impact. Avoiding activators such as TAED, and the like, means that the compositions of the Invention have an improved safety profile.

For the avoidance of doubt, reference to “% by weight” means by weight of the total composition, the amount of each ingredient being selected so that total ingredients in the composition sum to 100 percent by weight. For compositions of the Invention, the remainder or balance, is water. The term, “aqueous solution” refers to a solution wherein the solvent is water and other constituents of the composition are dispersed, mixed, and/or dissolved therein. Particularly, the compositions comprise at least 80% by weight of water, for example at least 85% by weight or 90% by weight of water. More particularly, the compositions may comprise at least 91%, at least 92%, at least 93%, at least 94%, at least 95%, at least 96%, at least 97%, at least 98% or at least 99% by weight of water. Thus, the compositions may comprise from 80% to 99% by weight of water, such as 85% to 99%, 90% to 99% or 95% to 99% by weight of water. Particularly water is the only solvent and organic solvents, such as alcohols, are not used.

Compositions of the invention comprise hydrogen peroxide, also referred to as H₂O₂ (CAS Number: 7722-84-)1. Particularly, compositions of the invention comprise from about 0.05% to about 4% by weight of hydrogen peroxide, such as from about 0.05% to about 4%, from about 0.1% to about 4% or from about 0.25% to about 4% by weight of hydrogen peroxide. More particularly, from about 0.05% to about 2%, from about 0.1% to about 2% or about 0.25% to about 2% by weight of hydrogen peroxide. Yet more particularly, from about 0.05% to about 1%, from about 0.1% to about 1% or from about 0.25% to about 1% by weight of hydrogen peroxide. Still yet more particularly, from about 0.1% to about 0.75% or from about 0.25% to about 0.75%, such as about 0.25%, about 0.3%, about 0.4%, about 0.5%, about 0.6%, about 0.7% or about 0.75% by weight of hydrogen peroxide.

Compositions of the invention further comprise at least one acid selected from the group consisting of salicylic acid, furoic acid and formic acid. Salicylic acid (CAS Number: 69-72-7) may also referred to as 2-Hydroxybenzoic acid. Formic acid (CAS Number: 64-18-6) may also be referred to as methanoic acid. Furoic acid refers to 2-Furoic acid (CAS Number: 88-14-2), also called Furan-2-carboxylic acid, and/or 3-Furoic acid (CAS Number: 488-93-7), also called Furan-3-carboxylic acid. The composition may comprise 2-Furoic acid and/or 3-Furoic acid. In some embodiments, the acid is 2-Furoic acid. In other embodiments, the acid is 3-Furoic acid.

In preferred embodiments of the invention, the acid is salicylic acid.

Whilst it is preferred to use one acid selected from the group consisting of salicylic acid, furoic acid and formic acid, a person skilled in the art will appreciate that combinations of these acids might also be used. For example, compositions of the invention further comprise at least two acids selected from the group consisting of salicylic acid, furoic acid and formic acid, for example two of these acids or three of these acids. Thus, in certain embodiments the compositions may comprise a combination of salicylic acid, furoic acid and formic acid, for example in any suitable molar ratio such as from 1:1:1 to 2:1:1, 1:2:1 or 1:1:2. In other embodiments, the compositions may comprise a combination of salicylic acid and furoic acid, a combination of salicylic acid and formic acid or a combination of furoic acid and formic acid, for example in any suitable molar ratio such as from 1:1 to 3:1, 1:1 to 1:1, such as 1:1, 1:2, 1:3, 3:1 or 2:1.

Compositions of the invention may comprise from about 0.01% to about 0.25% by weight of the acid, from about 0.1% to about 0.25% by weight of the acid or from about 0.15% to about 0.2% by weight of the acid, for example about 0.15%, 0.16%, 0.17%, 0.18%, 0.19% or about 0.2% by weight of the acid.

Compositions of the invention comprise a surfactant, more particularly an anionic surfactant. Suitable anionic surfactants are known in the art. Particularly, the anionic surfactant is a sodium alkyl sulfate, for example, a sodium sulfate having an alkyl group having 8 to 20 carbon atoms. Non-limiting examples of these include sodium decyl sulfate, sodium lauryl sulfate (also referred to as SLS or sodium dodecyl sulfate (SDS)), sodium tetradecyl sulfate, sodium hexadecyl sulfate and sodium sodium octadecyl sulfate. In certain embodiments, the anionic surfactant is a sodium alkyl sulfate selected from the group consisting of sodium lauryl sulfate, sodium lauryl sulfonate and sodium laureth sulfate. Particularly, the anionic surfactant is sodium lauryl sulfate.

The composition may comprise from about 0.05% to about 2.00% by weight of the anionic surfactant, particularly from about 0.1% to about 2% by weight, more particularly from about 0.25% to about 1% by weight or yet more particularly from about 0.3% to about 0.8% by weight of the anionic surfactant. For example, about 0.3%, 0.4%, 0.5%, 0.6%, 0.7% or about 0.8% by weight of the anionic surfactant.

Compositions of the invention preferably have a pH within the range of from 2.8 to 3.5, such as a pH within the range of from 2.8 to 3.2, for example a pH of about 3.0 or about 3.1. In order to achieve this, compositions of the invention may comprise a means for adjusting or maintaining the pH of the composition within this range. The composition may comprise from about 0.01% to about 0.09% by weight such as from about 0.02% to 0.08% by weight of the means for adjusting or maintaining the pH of the composition. The composition may comprise about 0.02%, 0.03%, 0.04%, 0.05%, 0.06%, 0.07% or about 0.08% by weight of the means for adjusting or maintaining the pH of the composition.

The means for adjusting or maintaining the pH of the composition may be an acid or a pH buffer (also referred to as a buffer). Particularly, the means for adjusting or maintaining the pH of the composition is a pH buffer. More particularly, the means for adjusting or maintaining the pH of the composition is a pH buffer comprising sodium citrate. Yet more particularly, the means for adjusting or maintaining the pH of the composition is a sodium citrate buffer. Sodium citrate buffers are known in the art and can be made and adjusted to the desired pH by mixing citric acid (monohydrate) and trisodium citrate (dehydrate), such as by way of non-limiting example, at ratios of from 82:18 to 73:27 respectively.

Particularly, compositions of the invention exhibit a dermal tolerance score of at least 10% mean cell viability, at least 11%, at least 12%, at least 13%, at least 14% or at least 15% mean cell viability as measured by in vitro dermal tolerance assay, more particularly as measured by the in vitro EPIDERM skin irritation test (EPI-200-SIT, MatTek Corporation).

Preferably, compositions of the invention are alcohol-free. The term “alcohol-free” refers to an absence of alcohol (0% by weight). For the avoidance of doubt, this term may include the presence of alcohol but in a quantity below the limit of detection of the analytical method and in an amount too low to be detected or accurately measured.

Alternatively, since some sensory components (discussed below) comprise alcohols, where a sensory component is included, compositions of the invention may be substantially alcohol-free. As used herein, “substantially alcohol free” means that the compositions contain less than 0.5% alcohol such as 0.4% or less, 0.3% or less, 0.25% or less, 0.2% or less, 0.1% or less, 0.05% or less or less than 0.01% alcohol.

Compositions of the invention are foamable formulations. A “foamable formulation” refers to a composition which is capable of forming a foam, particularly a foam having a small bubble size to give the appearance of a dense, rich foam The foam is capable of being brushed easily across a surface.

The compositions of the invention form a stable foam having desirable physicochemical properties such as volume, density, and spread that do not change significantly over a period of time such that the foam does not break down immediately during use. More particularly, foams formed by compositions of the invention are not “quick-breaking” and will be stable when dispensed from a container, for example by spraying, and not breakdown immediately in contact with a surface. Whilst foams formed by compositions of the invention will not be quick-breaking, they will be “breakable”, in other words, able to be spread easily across the surface with application of mild shear force. Characteristics of foams can be measured using tests known in the art, such as by conducting foam formation (foam height vs time) and foam collapsibility experiments. Preferably the foam retains its structure such that it remains in contact with the surface for a time sufficient to cleanse and/or kill micro-organisms. Particularly, the duration of use is 3 minutes or less, more particularly 2 minutes or less, yet more particularly 60 seconds or less, for example between about 30 and 60 seconds.

Compositions of the current invention are foamable in the absence of a propellant. Particularly compositions of the invention are dispensed from a container without use of a propellant. Particularly, the compositions are foamable by agitation, such as by shaking or stirring, or manual aeration, for example where atmospheric air is passed through the formulation to produce a foam such as by manual actuation of a dispensing head. Particularly, compositions of the invention may be dispensed from a container which has a suitable dispensing head, such as a foaming applicator head. The foam may be applied directly to the surface, optionally with brushing or wiping. After cleansing, the surface may optionally be rinsed with water or dry wiped, for example to remove any pools or droplets of the composition.

For compositions that are used to clean surfaces, it may be desirable to include at least one sensory component. A “sensory component” refers to a component that may be used to impart a fragrance, colour or taste to the composition. Other than imparting the compositions with desirable organoleptic properties, generally the sensory component is not an active component, in other words it generally does not play an active role in cleansing or bacterial killing. Particularly, the sensory component is at least one fragrance or perfume, colouring agent or bittering agent.

Suitable fragrances, also referred to as perfumes, include components that provide an olfactory benefit, for example masking any chemical odor associated with the product. The fragrance may improve the odor of the composition itself and/or impart an odor on a surface treated with a composition of the Invention. Fragrance ingredients are readily solubilized in the compositions of the Invention, for example by the detergent component. Suitable fragrance ingredients and compositions are known in the art and may be selected based on the desired scent and compatibility with the components of the composition, such as the hydrogen peroxide and acid.

A “colouring agent” also referred to as a “colourant” means any type of colour-imparting compound, such as a dye, pigment or other material. Particularly, any colouring agent is stable and not degraded by constituents of the composition such as hydrogen peroxide, the acid component or pH of the composition.

The term “bittering agent” as used herein refers to compounds used to impart an undesirable, bitter taste or flavor to deter animals, for example pets such as dogs and cats, or humans, for example children and babies, from ingesting (swallowing) the composition. Sensory components are known in the art and may be natural or artificial (synthetic).

Compositions of the invention may comprise from about 0.01% to about 0.5% by weight of a sensory component, particularly from about 0.1% to about 0.3% by weight of a sensory component, more particularly from about 0.2% to about 0.3% by weight of a sensory component, such as about 0.1%, 0.15%, 0.2%, 0.25%, 0.3%, 0.35%, 0.4%, 0.45% or about 0.5% by weight of a sensory component.

The sensory component must be compatible with the constituents of the composition and not change or degrade, for example, in contact with hydrogen peroxide or acid. More particularly, any fragrance(s), colouring agent(s) and bittering agent(s) used are peroxide-stable (i.e. stable in the presence of peroxide), acid-stable (i.e. stable in the presence of salicylic acid, furoic acid and formic acid) and pH-stable at a pH of from 2.8 to 3.5.

Typically, compositions of the invention are stable for at least 3 months at a room temperature of 20° C., particularly for at least 6 months at a room temperature of 20° C., more particularly for at least 12 months at a room temperature of 20° C. and yet more particularly for at least 24 months at a room temperature of 20° C.

As used herein, the term “stable” or “shelf-stable” refers to the ability of the composition to maintain certain physical or chemical properties, such as antimicrobial efficacy, during storage at a specific temperature for a period of time after production.

The stability of compositions of the present invention may be determined by comparing antimicrobial efficacy before and after storage for a period of time using a suitable killing or inactivation assay which measures the reduction in the number of live microorganisms present in a sample following exposure to the composition.

As used herein, the term “microorganism” refers to bacteria, fungi, such as yeasts or moulds, and viruses. Compositions of the Invention that kill and/or inhibit growth of bacteria, fungi and viruses and may be referred to as “antimicrobial” or “microbiocidal”. Thus, compositions of the Invention may be “biocidal” compositions, killing and/or inhibiting growth of bacteria and/or “fungicidal” compositions, killing and/or inhibiting growth of fungi and/or “virucidal” compositions, inactivating virus particles such that the virus particles are unable to infect host cells. Reference to “sanitising” refers to simultaneous cleaning and disinfection. As used herein, “disinfecting” refers to the killing of microorganisms living on a surface.

As used herein, the term “kill” refers to causing death of a microorganism. In relation to a population of microorganisms, the term “kill” or “killing” refers to reducing the total amount or level of living or viable microorganisms by causing the death of some or substantially all of the microorganisms in the population. With regard to viruses, kill or killing refers to rendering the virus incapable of infecting a host cell and/or rendering the virus incapable of replication in a host cell, for example by inactivation or destruction. Thus, the killing assay may be a bacterial killing assay, a fungal killing assay, a viral killing assay or viral inactivation assay.

In some embodiments, the composition is a virucidal composition capable of killing an influenza virus or a coronavirus. In certain embodiments the virus is influenza virus H1N1. In certain embodiments the virus is a coronavirus such as SARS-CoV, SARS-CoV-1 or SARS-CoV-2.

For the avoidance of doubt, use of the term “a” or “an” in relation to microorganisms such as bacteria, fungi and viruses is to be read to refer to one or more of that entity, for example a population of bacteria growing on a surface, such as in the form of a biofilm. The term “biofilm” refers to films formed of surface-attached communities of microorganisms encased within a self-produced extracellular polymeric matrix. Such biofilms often form in the corners and surfaces of aqueous environments such as, by way of non-limiting example, sinks, taps, drains and the like.

Opportunistic yeast species such as Candida albicans can be found everywhere in the environment including on foods such as citrous fruits, vegetables and breads. It is one of the few species of Candida that can infect humans causing candidiasis which results from an overgrowth of the fungus. C. albicans is commonly used as a model organism for fungal pathogens. Therefore, the efficacy and stability of compositions of the invention may be determined by measuring antimicrobial efficacy against Candida albicans using a suitable killing assay such as the assay described in the Examples. For example, stability may be determined by comparing the antimicrobial efficacy of a composition before and after a period of storage.

The level of killing may be defined as a proportion, such as a percentage, or by reference to a log reduction in the total amount or level of microorganisms. Log reduction is used in the art to refer to the number of live microorganisms killed, for example following contact of the microorganisms with the compositions of the Invention. The terms “log X reduction” and “x log reduction” are used interchangeably to refer to a reduction in the number of live microorganisms of 10^{x}. For example, a log 4 reduction means that after contacting a population of microorganisms with the composition, the number of remaining microorganisms in the population is 10,000 times less, i.e. 1/10⁴ or less of the population prior to contact with the composition.

Particularly, exposure of a suspension of Candida albicans (10⁸ cfu per ml) to a composition of the invention yields at least a log 2.5 reduction, particularly at least a log 3 reduction, more particularly at least a log 4 reduction, yet more particularly at least a log 5 reduction, in the number of Candida albicans colony forming units within 60 seconds of exposure when measured at least 3 months after preparation of the composition. Particularly, exposure of a suspension of Candida albicans (10⁸ cfu per ml) to a composition of the invention yields at least a log 2.5 reduction, particularly at least a log 3 reduction, more particularly at least a log 4 reduction, yet more particularly at least a log 5 reduction, in the number of Candida albicans colony forming units within 60 seconds of exposure when measured at least 6 months after preparation of the composition. Particularly, exposure of a suspension of Candida albicans (10⁸ cfu per ml) to a composition of the invention yields at least a log 2.5 reduction, particularly at least a log 3 reduction, more particularly at least a log 4 reduction, yet more particularly at least a log 5 reduction, in the number of Candida albicans colony forming units within 60 seconds of exposure when measured at least 12 months after preparation of the composition. Particularly, exposure of a suspension of Candida albicans (10⁸ cfu per ml) to a composition of the invention yields at least a log 2.5 reduction, particularly at least a log 3 reduction, more particularly at least a log 4 reduction, yet more particularly at least a log 5 reduction, in the number of Candida albicans colony forming units within 60 seconds of exposure when measured at least 24 months after preparation of the composition.

Klebsiella pneumoniae is a gram-negative bacteria that causes multiple healthcare-associated infections. It may often be found in domestic and healthcare settings, for example, on kitchen surfaces and sponges. Therefore, stability of compositions of the invention may also be determined by measuring antimicrobial efficacy against Klebsiella pneumoniae using a suitable bacterial killing assay such as the assay described in the Examples.

Particularly, exposure of a suspension of Klebsiella pneumoniae (10⁸ cfu per ml) to a composition of the invention yields at least a log 2.5 reduction, particularly at least a log 3 reduction, more particularly at least a log 4 reduction, yet more particularly at least a log 5 reduction, in the number of Klebsiella pneumoniae colony forming units within 60 seconds of exposure when measured at least 3 months after preparation of the composition. Particularly, exposure of a suspension of Klebsiella pneumoniae (10⁸ cfu per ml) to a composition of the invention yields at least a log 2.5 reduction, particularly at least a log 3 reduction, more particularly at least a log 4 reduction, yet more particularly at least a log 5 reduction, in the number of Klebsiella pneumoniae colony forming units within 60 seconds of exposure when measured at least 6 months after preparation of the composition. Particularly, exposure of a suspension of Klebsiella pneumoniae (10⁸ cfu per ml) to a composition of the invention yields at least a log 2.5 reduction, particularly at least a log 3 reduction, more particularly at least a log 4 reduction, yet more particularly at least a log 5 reduction, in the number of Klebsiella pneumoniae colony forming units within 60 seconds of exposure when measured at least 12 months after preparation of the composition. Particularly, exposure of a suspension of Klebsiella pneumoniae (10⁸ cfu per ml) to a composition of the invention yields at least a log 2.5 reduction, particularly at least a log 3 reduction, more particularly at least a log 4 reduction, yet more particularly at least a log 5 reduction, in the number of Klebsiella pneumoniae colony forming units within 60 seconds of exposure when measured at least 24 months after preparation of the composition.

Compositions of the invention retain at least 80%, at least 85%, at least 90%, at least 91%, at least 92%, at least 93%, at least 94%, at least 95%, at least 96%, at least 97%, at least 98% or at least 99% of antimicrobial efficacy after at least three months, after at least six months, at least twelve months or at least twenty-four months at room temperature (20° C.).

There is also provided a container comprising a composition of the invention. There is also provided a dispenser comprising (i) a container comprising a composition of the invention and (ii) an applicator head. More particularly, there is provided a dispenser consisting essentially of (i) a container comprising a composition of the invention and (ii) an applicator head configured to dispense the composition in the form of a spray and/or foam. Yet more particularly, there is provided a dispenser comprising or consisting essentially of, a container defining a reservoir and an applicator head in fluid communication with the reservoir, wherein the reservoir comprises a composition of the invention and wherein the applicator head is configured to dispense the composition from the reservoir in the form of a spray and/or foam.

The container may be manufactured from any suitable material. Suitable materials are those materials that do not degrade, interact, or react with the components of the composition. Particularly, the suitable material does not interact, react with or degrade on contact with hydrogen peroxide, more particularly on contact, such as prolonged contact, with 0.05% to about 4% by weight of hydrogen peroxide. Particularly, the suitable material does not interact, react with or degrade on contact, such as prolonged contact, with salicylic acid, furoic acid or formic acid, more particularly from 0.01% to about 0.25% by weight of salicylic acid, fuoric acid or formic acid. Particularly, the suitable material does not interact, react with or degrade on contact, such as prolonged contact, with an anionic surfactant, more particularly from 0.05% to about 2.00% by weight of the anionic surfactant.

By way of non-limiting example, the container may comprise high density polyethylene (HDPE) and/or polyethylene terephthalate (PET). In some embodiments, the container comprises high density polyethylene (HDPE) and polyethylene terephthalate (PET). In some embodiments, the container is a high-density polyethylene (HDPE) container. In some embodiments, the container is a polyethylene terephthalate (PET) container.

After accelerated stability testing of compositions of the invention stored in containers manufactured from high density polyethylene (HDPE) or polyethylene terephthalate (PET) and other plastic and metal components, no evidence of corrosion or other damage to the containers was noted. Thus, there is also provided a container comprising a composition of the invention, wherein the container is manufactured from polyethylene terephthalate (PET) or high-density polyethylene (HDPE). There is also provided a dispenser comprising (i) a container comprising a composition of the invention and (ii) an applicator head, wherein the container is manufactured from polypropylene (PP) More particularly, a dispenser consisting essentially of (i) a polyethylene terephthalate (PET) or high-density polyethylene (HDPE) container comprising a composition of the invention and (ii) an applicator head configured to dispense the composition in the form of a spray and/or foam. Yet more particularly, there is provided a dispenser comprising or consisting essentially of a polyethylene terephthalate (PET) or high-density polyethylene (HDPE) container defining a reservoir and an applicator head in fluid communication with the reservoir, wherein the reservoir comprises a composition of the invention and wherein the foaming applicator head is configured to dispense the composition from the reservoir in the form of a spray and/or foam.

Particularly, compositions of the Invention do not contain persalts such as sodium percarbonate, sodium perborate, sodium persulphate, potassium persulphate or ammonium persulphate. Use of the term “persalt” refers to any solid inorganic compound which liberates hydrogen peroxide and a salt of an inorganic acid when dissolved in water under normal conditions of temperature and pressure. Thus, compositions of the invention are sodium percarbonate free, sodium perborate free, sodium persulphate free, potassium persulphate free and/or ammonium persulphate free. Yet more particularly, compositions of the Invention are substantially free of persalts. The term “substantially free of persalts” refers to an absence of persalts (0% by weight). For the avoidance of doubt, this term may also include the presence of persalts but in a quantity below the limit of detection of the analytical method and in an amount too low to be detected or accurately measured.

Particularly, compositions of the Invention do not comprise complex peroxyacid bleach precursors such as N,N,N′,N′-tetraacetyl ethylene diamine (TAED), decanoyloxybenzoicacid (DOBA), decanoyloxybenzene sulphonate (DOBS), tetraacetyl glycoluril (TAGU), sodium acetoxybenzene sulphonate (SABS), nonanoyloxybenzene sulphonate (NOBS), sodium 4-(isononanoyloxy)benzenesulphonate (iso-NOBS), sodium nonanoyloxybenzene sulphonate (SNOBS) and sodium benzoyloxybenzene sulphonate (SBOBS), 4-(2-decanoyloxyethoxycarbonyloxy) benzenene sulfonate sodium (DECOBS) and lauroyloxybenzenesulfonatesodium (LOBS). Thus, compositions of the Invention are TAED-free, DOBA-free, DOBS-free, SABS-free, NOBS-free, iso-NOBS-free, SNOBS-free, DECOBS-free and LOBS-free.

### Compositions for Use in Cleansing

The present invention is also directed toward the compositions as described above for use in methods of cleaning surfaces, such as hard surfaces and/or soft surfaces.

These methods comprise applying the composition to a surface and leaving the composition in contact with the surface for a period of time.

The composition may be applied in the form of a spray and/or foam. Thus, the method may comprise applying the composition to a surface in the form of a spray and/or foam. The method may comprise dispensing the composition from a container in the form of a spray and/or foam. Particularly, when being dispensed, the composition may be activated to a foam via a foaming applicator head. The composition may be applied directly to the surface, such as a hard or soft surface, by spraying, optionally with wiping or agitation such as brushing. In some embodiments, the composition may be applied directly to the surface, such as a hard or soft surface, by pouring, optionally with wiping or agitation such as brushing.

In other embodiments, the composition may be applied to the surface, such as a hard or soft surface, using a substrate such as a sponge, wipe, cloth, brush and the like. In these embodiments, the composition would first be dispensed onto the substrate, for example by pouring or spraying, and then applied to the surface using the substrate, for example by wiping or brushing. In this manner the composition is transferred from the substrate onto the surface, particularly as a result of physical contact between the substrate and the surface. Similarly, the composition may be applied to the surface using a disposable premoistened pad which comprises a substrate impregnated with a cleansing composition of the invention. In such embodiments, the composition is applied to the surface using the disposable premoistened pad, for example by wiping or brushing, to transfer the composition from the substrate onto the surface.

Particularly, the composition is maintained in contact with the surface for a period of time.

Particularly, the composition is maintained in contact with the surface for a period of time sufficient to kill at least 80%, such as at least 85%, at least 90%, at least 95%, at least 96%, at least 97%, at least 98%, at least 99% or at least 99.9% of microorganisms present on the surface.

Particularly the microorganisms are Candida albicans and/or Klebsiella pneumoniae, more particularly Candida albicans. Particularly, the composition is maintained in contact with the surface for a period of time sufficient to achieve at least a log 2.5 reduction in the number of Candida albicans and/or Klebsiella pneumoniae colony forming units on the surface, preferably at least a log 3 reduction, more preferably at least a log 4 reduction, most preferably at least a log 5 reduction. Preferably, the period of time is no more than 3 minutes, 3 minutes or less, no more than 2 minutes, 2 minutes or less, no more than 60 seconds, 60 seconds or less, particularly from 30 seconds to 60 seconds. After this period of time, the surface may be wiped, for example with a cloth or sponge, or rinsed with water to remove residual composition.

The compositions of the Invention do not irritate the mucous membranes or eyes of a user, for example when sprayed onto a surface.

Compositions of the invention have improved dermal tolerance (for example, in comparison to certain other cleansers), and are particularly suitable for cleansing surfaces where the composition may come into contact with the skin of the hands or fingers, either accidentally or during manual handling steps. Particularly, compositions of the invention have a dermal tolerance score of at least 10% mean cell viability, at least 11%, at least 12%, at least 13%, at least 14% or at least 15% mean cell viability as measured by in vitro dermal tolerance assay.

In certain embodiments, there is provided a composition of the invention for use in a method of cleaning a surface, the method comprising: dispensing the composition from a container onto a surface, optionally spreading the composition across the surface, maintaining the composition in contact with the surface for a period of time, particularly from 30 to 60 seconds, and optionally wiping and/or rinsing the surface with water to remove residual composition.

In certain embodiments, there is provided a composition of the invention for use in a method of cleaning a surface, the method comprising: dispensing the composition from a container onto a surface in the form of a spray and/or foam, optionally spreading the composition across the surface, maintaining the composition in contact with the surface for a period of time, particularly from 30 to 60 seconds, optionally wiping and/or rinsing the surface with water to remove residual composition.

In certain embodiments, there is provided a composition of the invention for use in a method of cleaning a surface, the method comprising: dispensing the composition from a container onto a substrate, applying the composition to a surface using the substrate, maintaining the composition in contact with the surface for a period of time, particularly from 30 to 60 seconds, and optionally wiping and/or rinsing the surface with water to remove residual composition.

### Compositions for Use in Laundry

Compositions of the Invention may be used for cleaning soft surfaces such as textiles, typically prior to or during a washing cycle.

The compositions may aid the removal of dirt and stains from textiles. For example, compositions may decolorise dirt/stains either in solution or on textile fibres by bleaching and/or chemical degradation. Compositions may also disrupt the bonds between dirt/stain and the textile, lowering adherence, thereby making dirt and stains easier to remove or disperse during a subsequent washing cycle. Compositions of the Invention also provide a sanitising or disinfecting effect.

Thus, in some embodiments, compositions of the invention may be used to pre-treat textiles, such as clothes. In these embodiments, the composition is applied directly to the textile and permitted to remain in contact with the textile for a period of time before the textile is washed and/or rinsed during a subsequent washing cycle.

In some embodiments, compositions of the invention may be used to pre-treat stains on textiles, such as clothes. In these embodiments, the composition is applied directly to the stain and permitted to remain in contact with the stain for a period of time before the textile is washed and/or rinsed during a subsequent washing cycle.

In some embodiments, compositions of the invention may be used as a laundry booster. The term, “laundry booster” refers to an additive used to supplement a laundry detergent during a pre-wash, wash and/or rinse cycle of a laundering process. As an addition to a laundry detergent in a wash liquor, compositions of the Invention may improve, supplement or add to the effects of the detergent. When used as a laundry booster, compositions of the invention provide a sanitising or disinfecting effect. These effects may be particularly advantageous when cleaning baby clothes and nappies.

In some embodiments, the composition is used as a laundry booster to supplement the laundry detergent during the wash cycle only. In other embodiments, the composition is used as a laundry booster during the rinse cycle only.

Thus, there is provided a composition for use as a laundry booster in a method comprising: (i) forming a wash solution comprising the composition of the Invention, a laundry detergent composition, and water; contacting a textile with the wash solution; and subjecting the textile to a laundry wash cycle in the wash solution.

The laundering process may be a manual process, such as a handwash, or may be an automated process using a washing machine.

### Compositions for Use in Killing Microorganisms

There is also provided a composition of the invention, for use in a method of killing microorganisms, such as Candida albicans, on a surface. In some embodiments, there is provided a composition of the invention, for use in a method of killing bacteria, such as Klebsiella pneumoniae, on a surface. In some embodiments, there is provided a composition of the invention, for use in a method of killing fungus, for example yeast, such as Candida albicans, on a surface. In some embodiments, there is provided a composition of the invention, for use in a method of inactivating, for example killing, a virus on a surface.

These methods comprise applying the composition of the invention to a surface, said surface comprising at least one microorganism, and maintaining the composition in contact with the surface for a period of time sufficient to kill the at least one microorganism. A person skilled in the art will recognize that when the composition is applied to a surface that comprises microorganisms, the composition will also contact the microorganisms on the surface.

Particularly, the composition is maintained in contact with a surface for a period of time sufficient to kill at least 80%, such as at least 85%, at least 90%, at least 95%, at least 96%, at least 97%, at least 98%, at least 99% or at least 99.9% of microorganisms present on the surface.

Thus, the methods may comprise applying the composition of the invention to a surface, said surface comprising a population of microorganisms, and maintaining the composition in contact with the surface for a period of time sufficient to kill at least 80% of the population of microorganisms.

Particularly, the microorganisms are Candida albicans and/or Klebsiella pneumoniae, more particularly Candida albicans.

Particularly, the composition is maintained in contact with a surface for a period of time sufficient to achieve at least a log 2.5 reduction in the number of Candida albicans and/or Klebsiella pneumoniae colony forming units on the surface, preferably at least a log 3 reduction, more preferably at least a log 4 reduction, most preferably at least a log 5 reduction.

With regard to viruses, particularly the compositions of the Invention are capable of decreasing the viral titer (corresponding to the content of infectious viruses present per unit volume in a cell culture lysate) of a viral strain by at least log 4. A log 4 reduction corresponds to a 10⁴-fold or 99.99% reduction of the viral strain tested. The virus may be an enveloped or non-enveloped virus. The virus may be an influenza virus, a coronavirus or a rhinovirus. In certain embodiments, the virus is respiratory syncytial virus. In certain embodiments the virus is influenza virus H1N1. In certain embodiments the virus is a coronavirus such as SARS-CoV, SARS-CoV-1 or SARS-CoV-2.

Thus, the methods may comprise applying the composition of the invention to a surface, said surface comprising a population of bacteria and/or fungi, including for example Candida albicans and/or Klebsiella pneumoniae, and maintaining the composition in contact with the surface for a period of time sufficient to achieve a reduction in the number of colony forming units of bacteria and/or fungi, for example Candida albicans and/or Klebsiella pneumoniae, of at least 80% or equivalent to at least log 2.5.

Preferably, the period of time is no more than 3 minutes, 3 minutes or less, no more than 2 minutes, 2 minutes or less, no more than 60 seconds, 60 seconds or less, particularly from 30 seconds to 60 seconds. After this period of time, the surface may be wiped, for example with a cloth or sponge, and/or rinsed with water to remove any residual composition. Particularly, compositions of the invention have a dermal tolerance score of at least 10% mean cell viability, at least 11%, at least 12%, at least 13%, at least 14% or at least 15% mean cell viability as measured by in vitro dermal tolerance assay.

In certain embodiments, there is provided a composition of the invention for use in a method of killing microorganisms, such as Candida albicans, on a surface, the method comprising: dispensing the composition from a container onto a surface, said surface comprising microorganisms, maintaining the composition in contact with the surface for a period of time sufficient to kill the microorganisms, particularly from 30 to 60 seconds, and optionally wiping and/or rinsing the surface to remove the composition.

Particularly, the surface comprises a population of microorganisms, such as Candida albicans. More particularly, the surface is maintained in contact with the composition for a period of time sufficient to kill at least 80%, such as at least 85%, at least 90%, at least 95%, at least 96%, at least 97%, at least 98%, at least 99% or at least 99.9% of microorganisms present on the surface. Yet more particularly, at least 80%, such as at least 85%, at least 90%, at least 95%, at least 96%, at least 97%, at least 98%, at least 99% or at least 99.9% of microorganisms present on the surface are killed within 30 to 60 seconds. Still yet more particularly, at least 80%, such as at least 85%, at least 90%, at least 95%, at least 96%, at least 97%, at least 98%, at least 99% or at least 99.9% of Candida albicans CFUs present on the surface are killed within 30 to 60 seconds.

In certain embodiments, there is provided a composition of the invention for use in a method of killing microorganisms, such as Candida albicans, on a surface, the method comprising: applying the composition to a surface, said surface comprising microorganisms, maintaining the composition in contact with the surface for a period of time sufficient to kill the microorganisms, particularly from 30 to 60 seconds, and optionally wiping and/or rinsing the surface.

### Use of Compositions in Methods of Cleansing

The present invention further includes methods of cleansing a surface using a composition as described above.

For example, there is provided a method of cleansing a surface, the method comprising: applying the composition to a surface, maintaining the composition in contact with the surface for a period of time, particularly from 30 to 60 seconds, and optionally wiping and/or rinsing the surface.

In some embodiments, there is provided a method of cleansing a surface, the method comprising: applying a composition of the Invention to a surface in the form of a spray or foam, maintaining the composition in contact with the surface for a period of time, particularly from 30 to 60 seconds, wiping and/or rinsing the surface to remove the composition.

### Use of Compositions in Methods of Killing Microorganisms

There is also provided a method of killing microorganisms, such as Candida albicans, on a surface using a composition of the invention.

There is further provided a method of killing microorganisms, such as Candida albicans, on a surface, the method comprising: applying a composition of the Invention to a surface, said surface comprising microorganisms, maintaining the composition in contact with the surface for a period of time sufficient to kill the microorganisms, particularly from 30 to 60 seconds, and optionally wiping and/or rinsing the surface to remove the composition.

There is further provided a method of killing microorganisms, such as Candida albicans, on a surface, the method comprising: dispensing a composition of the invention onto a surface in the form of a spray or foam, said surface comprising microorganisms, maintaining the composition in contact with the composition for a period of time sufficient to kill the bacteria, particularly from 30 to 60 seconds, and optionally wiping and/or rinsing the surface.

There is also provided a disposable premoistened sponge or pad for cleaning hard and/or soft surfaces comprising: (i) a substrate and (ii) a composition of the invention, wherein the substrate is impregnated with the composition. The substrate may comprise cellulose, for example having a cellulose content of 80 gsm (Grams per Square Meter) to 200 gsm.

### GENERAL

The term “comprising” encompasses “including” e.g. a composition “comprising” X may include something additional e.g. X+Y. In some implementations, the term “comprising” refers to the inclusion of the indicated active agent, such as recited compounds, as well as inclusion of other active agents, and carriers, excipients, emollients, stabilizers, etc., known in the consumer health industry and generally recognized as safe (GRAS). Use of the transitional phrase “consisting essentially of” means that the scope of a claim is to be interpreted to encompass the specified materials or steps recited in the claim, and those that do not materially affect the basic and novel characteristic(s) of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising”. The term “consisting of” and variations thereof means including and limited to (for example, the specific recited constituents or steps). In certain territories, the term “comprising an active ingredient consisting of” may be used in place of “consisting essentially”. The term “about” in relation to a numerical value x is optional and means, for example, that the numerical value may comprise some variance around the stated number to allow for routine experimental fluctuation, measurement variance or to encompass minor deviations that may achieve substantially the same results as the stated number, such as x±10%, x±5%, x±4%, x±3%, x±2% or x±1%. The word “substantially” does not exclude “completely” e.g. a composition which is “substantially free” from Y may be completely free from Y. Where necessary, the word “substantially” may be omitted from the definition of the invention. Where methods refer to process steps, for example as (a), (b), (c), etc., these are intended to be sequential, i.e., step (c) follows step (b) which is preceded by step (a). All percentages and ratios used herein are by weight of total composition, unless otherwise indicated.

All references or patent applications cited within this patent specification are incorporated by reference herein.

### EMBODIMENTS OF THE INVENTION

The following clauses describe specific embodiments of the invention:
E1) An aqueous composition for cleaning hard and/or soft surfaces comprising: from about 0.05% to about 4% by weight of hydrogen peroxide, from about 0.01% to about 0.25% by weight of at least one acid selected from the group consisting of salicylic acid, furoic acid and formic acid, from about 0.05% to about 2.00% by weight of an anionic surfactant.
E2) An aqueous composition for cleaning hard and/or soft surfaces comprising or consisting essentially of: from about 0.05% to about 4% by weight of hydrogen peroxide, from about 0.01% to about 0.25% by weight of at least one acid selected from the group consisting of salicylic acid, furoic acid and formic acid, from about 0.05% to about 2.00% by weight of an anionic surfactant wherein the composition has a pH within the range of from 2.8 to 3.5.
E3) An aqueous composition for cleaning hard and/or soft surfaces comprising or consisting essentially of: from about 0.05% to about 4% by weight of hydrogen peroxide, from about 0.01% to about 0.25% by weight of at least one acid selected from the group consisting of salicylic acid, furoic acid and formic acid, from about 0.05% to about 2.00% by weight of an anionic surfactant, from about 0.01% to about 0.08% by weight of a buffer particularly, a sodium citrate buffer, wherein the composition has a pH within the range of from 2.8 to 3.5.
E4) An aqueous composition for cleaning hard and/or soft surfaces comprising or consisting essentially of: from about 0.05% to about 4% by weight of hydrogen peroxide, from about 0.01% to about 0.25% by weight of an acid selected from the group consisting of salicylic acid, furoic acid and formic acid, from about 0.05% to about 2.00% by weight of an anionic surfactant, from about 0.01% to about 0.08% by weight of a buffer particularly, a sodium citrate buffer, wherein the composition has a pH within the range of from 2.8 to 3.5 and wherein the composition further comprises from about 0.01% to about 0.5% by weight of a sensory component.
E5) An aqueous composition for cleaning hard and/or soft surfaces comprising or consisting essentially of: from about 0.05% to about 4% by weight of hydrogen peroxide, from about 0.01% to about 0.25% by weight of 2-furoic acid and/or 3-furoic acid, from about 0.05% to about 2.00% by weight of an anionic surfactant wherein the composition has a pH within the range of from 2.8 to 3.5.
E6) An aqueous composition for cleaning hard and/or soft surfaces comprising or consisting essentially of: from about 0.05% to about 4% by weight of hydrogen peroxide, from about 0.01% to about 0.25% by weight of formic acid, from about 0.05% to about 2.00% by weight of an anionic surfactant wherein the composition has a pH within the range of from 2.8 to 3.5.
E7) An aqueous composition for cleaning hard and/or soft surfaces comprising or consisting essentially of: from about 0.05% to about 4% by weight of hydrogen peroxide, from about 0.01% to about 0.25% by weight of salicylic acid, from about 0.05% to about 2.00% by weight of an anionic surfactant wherein the composition has a pH within the range of from 2.8 to 3.5.
E8) An aqueous composition for cleaning hard and/or soft surfaces comprising or consisting essentially of: from about 0.1% to about 2% by weight of hydrogen peroxide, from about 0.01% to about 0.25% by weight of salicylic acid, from about 0.05% to about 2.00% by weight of an anionic surfactant wherein the composition has a pH within the range of from 2.8 to 3.5.
E9) An aqueous composition for cleaning hard and/or soft surfaces comprising or consisting essentially of: from about 0.1% to about 2% by weight of hydrogen peroxide, from about 0.01% to about 0.25% by weight of salicylic acid, from about 0.25% to about 1.00% by weight of an anionic surfactant wherein the composition has a pH within the range of from 2.8 to 3.5.
E10) An aqueous composition for cleaning hard and/or soft surfaces comprising or consisting essentially of: from about 0.1% to about 2% by weight of hydrogen peroxide, from about 0.1% to about 0.2% by weight of salicylic acid, from about 0.25% to about 1.00% by weight of an anionic surfactant wherein the composition has a pH within the range of from 2.8 to 3.5.
E11) An aqueous composition for cleaning hard and/or soft surfaces comprising or consisting essentially of: from about 0.25% to about 1% by weight of hydrogen peroxide, from about 0.01% to about 0.25% by weight of salicylic acid, from about 0.05% to about 2.00% by weight of an anionic surfactant wherein the composition has a pH within the range of from 2.8 to 3.5.
E12) An aqueous composition for cleaning hard and/or soft surfaces comprising or consisting essentially of: from about 0.25% to about 1% by weight of hydrogen peroxide, from about 0.1% to about 0.2% by weight of salicylic acid, from about 0.25% to about 1.00% by weight of an anionic surfactant wherein the composition has a pH within the range of from 2.8 to 3.5.
E13) An aqueous composition for cleaning hard and/or soft surfaces comprising or consisting essentially of: from about 0.05% to about 4% by weight of hydrogen peroxide, from about 0.01% to about 0.25% by weight of salicylic acid, from about 0.05% to about 2.00% by weight of an anionic surfactant, from about 0.01% to about 0.08% by weight of a buffer particularly, a sodium citrate buffer, wherein the composition has a pH within the range of from 2.8 to 3.5.
E14) An aqueous composition for cleaning hard and/or soft surfaces comprising or consisting essentially of: from about 0.1% to about 2% by weight of hydrogen peroxide, from about 0.1% to about 0.2% by weight of salicylic acid, from about 0.25% to about 1.00% by weight of an anionic surfactant, from about 0.01% to about 0.08% by weight of a buffer particularly, a sodium citrate buffer wherein the composition has a pH within the range of from 2.8 to 3.5.
E15) An aqueous composition for cleaning hard and/or soft surfaces comprising or consisting essentially of: from about 0.1% to about 2% by weight of hydrogen peroxide, from about 0.1% to about 0.2% by weight of salicylic acid, from about 0.25% to about 1.00% by weight of an anionic surfactant, from about 0.02% to about 0.06% by weight of a buffer particularly, a sodium citrate buffer wherein the composition has a pH within the range of from 2.8 to 3.5.
E16) An aqueous composition for cleaning hard and/or soft surfaces comprising or consisting essentially of: from about 0.1% to about 2% by weight of hydrogen peroxide, from about 0.1% to about 0.2% by weight of salicylic acid, from about 0.25% to about 1.00% by weight of an anionic surfactant, from about 0.02% to about 0.08% by weight of a sodium citrate buffer wherein the composition has a pH within the range of from 2.8 to 3.5.
E17) An aqueous composition for cleaning hard and/or soft surfaces comprising or consisting essentially of: from about 0.25% to about 1% by weight of hydrogen peroxide, from about 0.1% to about 0.2% by weight of salicylic acid, from about 0.25% to about 1.00% by weight of an anionic surfactant, from about 0.01% to about 0.08% by weight of a buffer particularly, a sodium citrate buffer wherein the composition has a pH within the range of from 2.8 to 3.5.
E18) An aqueous composition for cleaning hard and/or soft surfaces comprising or consisting essentially of: from about 0.25% to about 1% by weight of hydrogen peroxide, from about 0.1% to about 0.2% by weight of salicylic acid, from about 0.25% to about 1.00% by weight of an anionic surfactant, from about 0.02% to about 0.08% by weight of a buffer particularly, a sodium citrate buffer wherein the composition has a pH within the range of from 2.8 to 3.5.
E19) An aqueous composition for cleaning hard and/or soft surfaces comprising or consisting essentially of: from about 0.25% to about 1% by weight of hydrogen peroxide, from about 0.1% to about 0.2% by weight of salicylic acid, from about 0.25% to about 1.00% by weight of an anionic surfactant, from about 0.02% to about 0.08% by weight of a sodium citrate buffer wherein the composition has a pH within the range of from 2.8 to 3.5.
E20) An aqueous composition for cleaning hard and/or soft surfaces comprising or consisting essentially of: from about 0.05% to about 4% by weight of hydrogen peroxide, from about 0.01% to about 0.25% by weight of salicylic acid, from about 0.05% to about 2.00% by weight of an anionic surfactant, from about 0.01% to about 0.08% by weight of a buffer particularly, a sodium citrate buffer, wherein the composition has a pH within the range of from 2.8 to 3.5 and wherein the composition further comprises from about 0.01% to about 0.5% by weight of a sensory component.
E21) An aqueous composition for cleaning hard and/or soft surfaces comprising or consisting essentially of: from about 0.1% to about 2% by weight of hydrogen peroxide, from about 0.1% to about 0.2% by weight of salicylic acid, from about 0.25% to about 1.00% by weight of an anionic surfactant, from about 0.02% to about 0.08% by weight of a sodium citrate buffer wherein the composition has a pH within the range of from 2.8 to 3.5 and wherein the composition further comprises from about 0.01% to about 0.5% by weight of a sensory component.
E22) An aqueous composition for cleaning hard and/or soft surfaces comprising or consisting essentially of: from about 0.1% to about 2% by weight of hydrogen peroxide, from about 0. 1% to about 0.2% by weight of salicylic acid, from about 0.25% to about 1.00% by weight of an anionic surfactant, from about 0.02% to about 0.08% by weight of a sodium citrate buffer wherein the composition has a pH within the range of from 2.8 to 3.5 and wherein the composition further comprises from about 0.1% to about 0.3% by weight of a sensory component.
E23) An aqueous composition for cleaning hard and/or soft surfaces comprising or consisting essentially of: from about 0.25% to about 1% by weight of hydrogen peroxide, from about 0.1% to about 0.2% by weight of salicylic acid, from about 0.25% to about 1.00% by weight of an anionic surfactant, from about 0.02% to about 0.08% by weight of a sodium citrate buffer wherein the composition has a pH within the range of from 2.8 to 3.5 and wherein the composition further comprises from about 0.01% to about 0.5% by weight of a sensory component.
E24) An aqueous composition for cleaning hard and/or soft surfaces comprising or consisting essentially of: from about 0.25% to about 1% by weight of hydrogen peroxide, from about 0.1% to about 0.2% by weight of salicylic acid, from about 0.25% to about 1.00% by weight of an anionic surfactant, from about 0.02% to about 0.08% by weight of a sodium citrate buffer wherein the composition has a pH within the range of from 2.8 to 3.5 and wherein the composition further comprises from about 0.1% to about 0.3% by weight of a sensory component.
E25) An aqueous composition for cleaning hard and/or soft surfaces comprising or consisting essentially of: from about 0.25% to about 1% by weight of hydrogen peroxide, from about 0.1% to about 0.2% by weight of salicylic acid, from about 0.25% to about 1.00% by weight of sodium lauryl sulfate, from about 0.02% to about 0.08% by weight of a sodium citrate buffer wherein the composition has a pH within the range of from 2.8 to 3.5 and wherein the composition further comprises from about 0.1% to about 0.3% by weight of a sensory component.
E26) An aqueous composition for cleaning hard and/or soft surfaces comprising or consisting essentially of: from about 0.25% to about 1% by weight of hydrogen peroxide, from about 0.01% to about 0.25% by weight of 2-furoic acid and/or 3-furoic acid, from about 0.25% to about 1.00% by weight of an anionic surfactant, from about 0.02% to about 0.08% by weight of a sodium citrate buffer wherein the composition has a pH within the range of from 2.8 to 3.5 and wherein the composition further comprises from about 0.1% to about 0.3% by weight of a sensory component.
E27) An aqueous composition for cleaning hard and/or soft surfaces comprising or consisting essentially of: from about 0.25% to about 1% by weight of hydrogen peroxide, from about 0.01% to about 0.25% by weight of 2-furoic acid and/or 3-furoic acid, from about 0.25% to about 1.00% by weight of sodium lauryl sulfate, from about 0.02% to about 0.08% by weight of a sodium citrate buffer wherein the composition has a pH within the range of from 2.8 to 3.5 and wherein the composition further comprises from about 0.1% to about 0.3% by weight of a sensory component.
E28) An aqueous composition for cleaning hard and/or soft surfaces comprising or consisting essentially of: from about 0.25% to about 1% by weight of hydrogen peroxide, from about 0.01% to about 0.25% by weight of formic acid, from about 0.25% to about 1.00% by weight of an anionic surfactant, from about 0.02% to about 0.08% by weight of a sodium citrate buffer wherein the composition has a pH within the range of from 2.8 to 3.5 and wherein the composition further comprises from about 0.1% to about 0.3% by weight of a sensory component.
E29) An aqueous composition for cleaning hard and/or soft surfaces comprising or consisting essentially of: from about 0.25% to about 1% by weight of hydrogen peroxide, from about 0.01% to about 0.25% by weight of formic acid, from about 0.25% to about 1.00% by weight of sodium lauryl sulfate, from about 0.02% to about 0.08% by weight of a sodium citrate buffer wherein the composition has a pH within the range of from 2.8 to 3.5 and wherein the composition further comprises from about 0.1% to about 0.3% by weight of a sensory component.
E30) A disposable premoistened sponge or pad for cleaning hard and/or soft surfaces comprising: (i) a substrate and (ii) a composition of the invention, wherein the substrate is impregnated with a composition according to any one of Embodiments E1 to E29.

In order that this invention may be better understood, the following examples are set forth. These examples are for purposes of illustration only and are not to be construed as limiting the scope of the invention in any manner.

### EXAMPLES

### Example 1

Aqueous compositions containing 0.5% hydrogen peroxide, 0.5% sodium lauryl sulfate, an acid (see Table 1 for amounts) and water (balance to 100%) were prepared and tested for in vitro anti-microbial efficacy against Candida albicans, an opportunistic pathogenic yeast.

Testing was conducted by adding the compositions to a planktonic suspension of Candida albicans (1-5×10⁸ cfu/ml) and then neutralising after 60 seconds contact time. The log reduction in Candida albicans was then calculated for each composition, with a performance target of>log 5 reduction (kills 99.999%).

TABLE 1
Anti-fungal efficacy of compositions containing
water, 0.5% hydrogen peroxide, 0.5% sodium lauryl
sulfate and different acids at pH 2.8-3.2

| | | | | Log Reduction vs |
|---|---|---|---|---|
| | Acid | Acid | Formulation | Candida albicans |
| Acid | w/w % | pKa | pH* | in 60 s |
| Phosphoric acid | 0.10 | 2.16 | 2.8 | <1.05 |
| Citric acid | 1.00 | 2.79 | 3.1 | <1.06 |
| Salicylic acid | 0.18 | 2.97 | 3.0 | >5.64 |
| Furoic acid | 0.24 | 3.12 | 3.1 | 2.70 |
| Formic acid | 0.08 | 3.74 | 3.0 | 2.50 |
| Oxalic acid | 0.20 | 3.81 | 2.8 | <1.05 |
| Lactic acid | 0.24 | 3.86 | 3.1 | <1.05 |
| Succinic acid | 0.24 | 4.21 | 3.2 | <1.10 |

*Buffered using sodium citrate where appropriate to achieve desired pH range

Of these compositions, only those comprising salicylic acid, furoic acid or formic acid were capable of achieving a log 2.50 or greater reduction in colony forming units of C. albicans. Surprisingly, compositions comprising 0.18% of salicylic acid yielded a>log 5 reduction (equivalent to 99.99% killing). Other acids tested achieved<1.10 log reduction demonstrating low levels of anti-microbial efficacy. There was no correlation between acid pKa and anti-bacterial efficacy.

### Example 2

The composition containing salicylic acid was then tested against Klebsiella pneumoniae, a gram-negative bacteria that causes multiple healthcare-associated infections.

TABLE 2
Anti-bacterial efficacy of compositions containing
0.5% hydrogen peroxide, 0.5% sodium lauryl sulfate,
0.18% salicylic acid and 98.82% water, pH 2.8-3.2

| | | | | Log Reduction vs |
|---|---|---|---|---|
| | Acid | Acid | Formulation | Klebsiella pneumoniae |
| Acid | w/w % | pKa | pH* | in 60 s |
| Salicylic acid | 0.18 | 2.97 | 3.0 | >6.44 |

These results confirmed the surprisingly strong anti-bacterial efficacy of compositions comprising salicylic acid.

### Example 3

Compositions containing water, 0.5% hydrogen peroxide, 0.5% sodium lauryl sulfate and salicylic acid were prepared at different pH's and placed into storage at 40° C. for 3 months to determine a suitable pH range for formulation stability.

Aged compositions were tested for in vitro antimicrobial efficacy against Candida albicans (60 s contact time as above), as well as foam appearance after dispensing the liquid composition through a foaming-pump packaging.

TABLE 3
Formulation stability of compositions containing water,
0.5% hydrogen peroxide, 0.5% sodium lauryl sulfate and
salicylic acid at different formulation pH ranges

| | Log Reduction vs | | | |
|---|---|---|---|---|
| | Candida albicans | | | |
| Formulation | in 60 s | | Dispensed foam appearance | |
| pH Range | Initial | Aged | Initial | Aged |
| <2.8 | >5.64 | >5.37 | Rich, compact | No foam |
| | | | foam | (hazy liquid) |
| 2.8-3.5 | >5.64 | >5.64 | Rich, compact | Rich, compact |
| | | | foam | foam |
| >3.5 | 3.76 | 2.45 | Rich, compact | Rich, compact |
| | | | foam | foam |

It was observed that a formulation pH of below pH 2.8 impacted foaming, resulted in a loss of foamability after sample aging. Without wishing to be bound by theory, it is believed that the sodium lauryl sulfate is degraded into insoluble lauryl alcohol below pH 2.8, resulting in a gradual loss of foamability over time.

It was also observed that formulations having a pH above 3.5 had a lower initial antimicrobial efficacy (3.76 log Reduction vs Candida albicans in 60 s) which declined during storage. Again, without wishing to be bound by theory, it is believed that the hydrogen peroxide begins to degrade at less acidic pH's, resulting in a gradual loss of efficacy over time.

Accordingly, it was determined that the optimum pH for stability and antimicrobial efficacy was within the range from pH 2.8 to pH 3.5.

### Example 4

A composition containing 0.5% hydrogen peroxide, 0.5% sodium lauryl sulfate, 0.18% salicylic acid and 98.82% water (pH 2.8-3.2) was prepared.

In vitro dermal tolerance was measured using the commercial in vitro EPIDERM skin irritation test (EPI-200-SIT, MatTek Corporation), also described in Kandarova et al (Pre-validation of an in vitro skin irritation test for medical devices using the reconstructed human tissue model EpiDerm™ Toxicol. In Vitro. 2018;50:407-417). In brief, a 3D tissue model of normal, human-derived epidermal keratinocytes (NHEK) were cultured and cell viability measured using a well-known cell viability assay. Cultures were then topically exposed to the cleanser solutions for a period of 60 seconds and cell viability re-measured to determine mean % cell viability post product exposure. A target remaining cell viability was set at 2.71%, representing an excellent dermal tolerance profile.

Material compatibility studies were conducted on a broad range of materials, including polymethyl methacrylate, polyamide, stainless steel, silver solder, ethylenevinyl acetate copolymer, thermoplastic polyurethane, cobalt-chromium alloy and polypropylene. Materials were repeatedly exposed to compositions for 60 seconds with brushing and then rinsing, over multiple cycles to represent 2 years simulated daily usage. Materials were then assessed using light microscopy and electron microscopy for surface damage. Material integrity post-exposure was visually graded as either structurally damaged (poor compatibility), minor superficially damaged (moderate/good compatibility) or not damaged (excellent compatibility).

TABLE 4
Comparison of anti-microbial kill time, dermal tolerance
and material compatibility profiles.

| | Claimed anti- | In vitro dermal | |
|---|---|---|---|
| | microbial | tolerance (% mean | Material |
| Cleanser Product | kill time | cell viability)** | compatibility* |
| Composition | 60 seconds | 15.26 | Excellent |
| of this invention | | | |

*Based on surface analysis of common materials after 2 years simulated product usage. Excellent = No/minimal change to material surface
**Using EPIDERM human epidermal model (MatTek, Inc.)

Surprisingly, the composition of the invention provided fast antimicrobial efficacy within 60 seconds (convenience) despite having a low-level of hydrogen peroxide (0.5%). The composition of the invention also provided excellent dermal tolerance (safety) and material compatibility.

### Example 5

The stain removal properties of the composition were evaluated in a simulated stain removal test. Ceramic tiles were stained with a concentrated tea solution. Half of the tile was treated with the composition, brushed, rinsed and dried (FIG. 1). Compositions of the invention effectively removed stains from ceramic surfaces.

In view of this guidance, one of ordinary skill in the art will be able to adjust the parameters provided in the Examples to achieve similar results within the remit of the claims.

### CLAIMS

1. An aqueous cleaning composition comprising: (a) from about 0.05% to about 4% by weight of hydrogen peroxide, (b) from about 0.01% to about 0.25% by weight of at least one acid selected from the group consisting of salicylic acid, fuoric acid and formic acid, (c) from about 0.05% to about 2.00% by weight of an anionic surfactant, and (d) from about 90% by weight of water, wherein the composition has a pH of from 2.8 to 3.5.
2. The composition of claim 1 further comprising (e) a means for adjusting or maintaining the pH of the composition.
3. The composition of claim 2, wherein the means for adjusting or maintaining the pH of the composition is a pH buffer.
4. The composition of claim 3 which comprises from about 0.01% to about 0.08% by weight of the pH buffer.
5. The composition of claim 3 or 4, wherein the pH buffer is sodium citrate buffer.
6. The composition of any one of claims 1 to 5, wherein the acid is salicylic acid.
7. The composition of any one of claims 1 to 6, which comprises from about 0.1% to about 2% by weight of hydrogen peroxide, particularly from about 0.25% to 1% by weight of hydrogen peroxide.
8. The composition of claim 6 or 7, wherein the anionic surfactant is a sodium alkyl sulfate.
9. The composition of claim 8, wherein the sodium alkyl sulfate is selected from the group consisting of sodium lauryl sulfate, sodium lauryl sulfonate and sodium laureth sulfate.
10. The composition of claim 1 which consists essentially of: (a) from about 0.1% to about 2% by weight of hydrogen peroxide, (b) from about 0.01% to about 0.25% by weight of salicylic acid, (c) from about 0.05% to about 2.00% by weight of an anionic surfactant selected from the group consisting of sodium lauryl sulfate, sodium lauryl sulfonate and sodium laureth sulfate, (d) at least 90% by weight of water and (e) a pH buffer, wherein the composition has a pH of from 2.8 to 3.5.
11. The composition of any preceding claim, further comprising (f) a sensory component, particularly a perfume, colouring agent and/or bittering agent.
12. The composition of any one of claims 1 to 11 for use in a method of killing microorganisms such as Candida albicans on a surface comprising, contacting the microorganisms on the surface with the composition for a period of time sufficient to kill the microorganisms.
13. A method of cleaning a hard surface comprising, contacting the hard surface with the composition of any one of claims 1 to 11, and optionally wiping the hard surface to spread and/or remove the composition.
14. A method of cleaning a soft surface comprising, contacting the soft surface with the composition of any one of claims 1 to 11, and optionally rinsing the surface to remove the composition.
15. A disposable premoistened pad for cleaning hard surfaces comprising: (i) a substrate and (ii) a composition according to any one of claims 1 to 11, wherein the substrate is impregnated with the composition.
16. The disposable premoistened pad of claim 15, for use in a method of cleansing a hard or soft surface.

1. An aqueous cleaning composition comprising: (a) from about 0.05% to about 4% by weight of hydrogen peroxide, (b) from about 0.01% to about 0.25% by weight of at least one acid selected from the group consisting of salicylic acid, fuoric acid and formic acid, (c) from about 0.05% to about 2.00% by weight of an anionic surfactant, and (d) from about 90% by weight of water, wherein the composition has a pH of from 2.8 to 3.5.

2. The composition of claim 1 further comprising (e) a means for adjusting or maintaining the pH of the composition.

3. The composition of claim 2, wherein the means for adjusting or maintaining the pH of the composition is a pH buffer.

4. The composition of claim 3 which comprises from about 0.01% to about 0.08% by weight of the pH buffer.

5. The composition of claim 3, wherein the pH buffer is sodium citrate buffer.

6. The composition of claim 1, wherein the acid is salicylic acid.

7. The composition of claim 1, which comprises from about 0.1% to about 2% by weight of hydrogen peroxide.

8. The composition of claim 6, wherein the anionic surfactant is a sodium alkyl sulfate.

9. The composition of claim 8, wherein the sodium alkyl sulfate is selected from the group consisting of sodium lauryl sulfate, sodium lauryl sulfonate and sodium laureth sulfate.

10. The composition of claim 1 which consists essentially of: (a) from about 0.1% to about 2% by weight of hydrogen peroxide, (b) from about 0.01% to about 0.25% by weight of salicylic acid, (c) from about 0.05% to about 2.00% by weight of an anionic surfactant selected from the group consisting of sodium lauryl sulfate, sodium lauryl sulfonate and sodium laureth sulfate, (d) at least 90% by weight of water and (e) a pH buffer, wherein the composition has a pH of from 2.8 to 3.5.

11. The composition of claim 1, further comprising (f) a sensory component, particularly a perfume, colouring agent and/or bittering agent.

12. The composition of claim 1, for use in a method of killing microorganisms such as Candida albicans on a surface comprising, contacting the microorganisms on the surface with the composition for a period of time sufficient to kill the microorganisms.

13. A method of cleaning a hard surface comprising, contacting the hard surface with the composition of claim 1, and optionally wiping the hard surface to spread and/or remove the composition.

14. A method of cleaning a soft surface comprising, contacting the soft surface with the composition of claim 1, and optionally rinsing the surface to remove the composition.

15. A disposable premoistened pad for cleaning hard surfaces comprising: (i) a substrate and (ii) a composition according to claim 1, wherein the substrate is impregnated with the composition.

16. The disposable premoistened pad of claim 15, for use in a method of cleansing a hard or soft surface.
